(19) United States
(12) Patent Application Publication
(10) Pub. No.: US 2008/0064630 A1
(43) Pub. Date: Mar. 13, 2008

(54) Osteogenic Growth Peptide Fusion Proteins

(75) Inventors: Niels Blume, Gilleleje (DK); Jing Su, Beijing (CN); Kjeld Madsen, Vaerlose (DK); Nils Langeland Johansen, Copenhagen O (DK); Lars Fogh Iversen, Holte (DK)

Correspondence Address: NOVO NORDISK, INC.;PATENT DEPARTMENT, 100 COLLEGE ROAD WEST, PRINCETON, NJ, 08540, US

(73) Assignee: NOVO NORDISK A/S, BAGSVAERD (DK)

(21) Appl. No.: 11/578,629

(22) PCT Filed: Apr. 14, 2005
(86) PCT No.: PCT/DK05/00256

§ 371 Date: Sep. 6, 2007

Related U.S. Application Data
(60) Provisional application No. 60/565,482, filed on Apr. 26, 2004.

(30) Foreign Application Priority Data
Apr. 19, 2004 (DK) PA 2004 00609

(51) Int. Cl. A61K 38/00; A61P 43/00; C07H 21/04; C07K 16/00; C12N 15/00; C12N 5/00; C12P 21/04

(52) U.S. Cl. 514/12; 435/320.1; 435/325; 435/69.7; 530/300; 530/350; 536/23.4

(57) ABSTRACT

Fusion protein comprising a protein to be fused, e.g. a therapeutic protein fused to the C-terminal of osteogenic growth peptide (OGP). The fusion proteins have a prolonged circulation time.

### FIELD OF THE INVENTION

The invention relates to osteogenic growth peptide (OGP) fusion proteins. The invention also relates to methods of increasing the circulation time of therapeutic proteins by fusing them to OGP or variants thereof, and to therapeutic method comprising the administration of OGP fusion proteins.

### BACKGROUND OF THE INVENTION

Osteogenic growth peptide (OGP) is a tetradecapeptide identical to the C-terminal amino acid sequence 89-102 of Histone H4. The amino acid sequence of OGP is Ala-Leu-Lys-Arg-Gln-Gly-Arg-Thr-Leu-Tyr-Gly-Phe-Gly-Gly (SEQ ID NO:33). OGP is a key factor in the mechanism of the systemic osteogenic response to local bone marrow injury. The plasma levels of OGP depend e.g. on age but it is present in a high abundance, up to 480-4460 μM. However, 80-90% of the OGP is non-covalently bound to other plasma proteins, the most important of which is α₂-macroglobulin (α₂M) [Biochem., 36, 14883-14888, 1997].

Bound OGP is inactive, but upon dissociation from α₂M and proteolytic cleavage, the biologically active, osteogenic OGP(10-14) is formed. This shows that the growth promoting activity is contained within the C-terminal fragment OGP(10-14), whereas the N-terminal fragment, OGP(1-9) is responsible for the α₂M binding.

Many attempts have been made to increase circulation time of proteins, and in particular therapeutically relevant proteins. Classically, the protein has been conjugated to e.g. fatty acids, which is believed to bind to albumin, or to large molecules, such as polyethylene glycol (PEG) which increase molecular size to decrease renal clearance [J. Pharm. Sci., 86, 1365-1368, 1997; U.S. Pat. No. 4,179,337]. Another approach is to fuse the protein of interest to another protein, such as albumin [U.S. Pat. No. 5,045,312; WO 97/24445; WO 01/79271].

A fusion protein of salmon calcitonin and OGP is disclosed in Zhongguo Shengwu Gongcheng Zazhi (2002), 22(4), 84-88, and it is reported that this fusion protein could increase the proliferation of osteoblastic and fibroblastic cells, stimulate the ALP activity and decrease the level of serum calcium in vitro and in vivo.

The number of known proteins with interesting biological or therapeutic activities is rapidly growing, inter alia as a result of the human genome project. However, the therapeutic potential of these novel proteins as well as “old” and well-established proteins is often limited by very short half-life in circulation. Hence, there remains a need for methods of increasing the circulation time of proteins in circulation, and for imparting other advantageous or alternative characteristics to such proteins (such as improved or alternative physiochemical properties).

### SUMMARY OF THE INVENTION

The present invention relates to OGP fusion proteins comprising a first protein fused to the C-terminus of OGP or a variant thereof (an “OGP variant”), optionally via a linker, provided that if said OGP or variant thereof is OGP then said protein is not salmon calcitonin, and provided that the fusion protein is not OGP itself.

In another embodiment, the invention relates to a method of increasing circulation time of OGP proteins in circulation, the method comprising fusing a protein (a “fusion partner”) to the C-terminal of OGP or to the C-terminal of a variant of OGP.

In another embodiment, the invention relates to a method of improving the physico-chemical properties of a protein, the method comprising fusing said protein, optionally via a linker, to the C-terminal of OGP or to the C-terminal of a variant of OGP.

In another embodiment, the invention relates to variants of OGP.

In another embodiment, the invention relates to nucleic acid constructs encoding the OGP fusion proteins or the OGP variants of the present invention, to vectors containing said nucleic acid constructs, to host cells transformed with said vectors, and methods of making the OGP fusion proteins and OGP variants of the present invention, provided that said OGP fusion protein is not salmon calcitonin fused directly to OGP or OGP itself, using these nucleic acids constructs, vectors and/or host cells.

In another embodiment, the invention relates to the use of OGP fusion proteins in therapy, provided said OGP fusion protein is not salmon cacitonin fused directly to OGP, or OGP.

In another embodiment, the invention relates to pharmaceutical compositions comprising an OGP fusion protein, provided said OGP fusion protein is not salmon cacitonin fused directly to OGP or OGP itself.

In a further embodiment, the invention relates to a transgenic organism modified to contain the nucleic acid construct of the present invention and to express OGP fusion proteins or OGP variants.

In a still further embodiment, the invention relates to therapeutic methods comprising the administration (or delivery, e.g., by expression from a recombinant nucleic acid) of a therapeutically effective amount of an OGP fusion protein to a patient in need thereof, provided said OGP fusion protein is not salmon calcitonin fused directly to OGP or OGP itself.

In a still further embodiment, the invention relates to the use of an OGP fusion protein in the manufacture of a medicament, provided said OGP fusion protein is not salmon calcitonin fused directly to OGP or OGP itself.

### DESCRIPTION OF THE DRAWINGS

FIG. 1: A feature map of the parental pNNC19 bacterial expression vector.

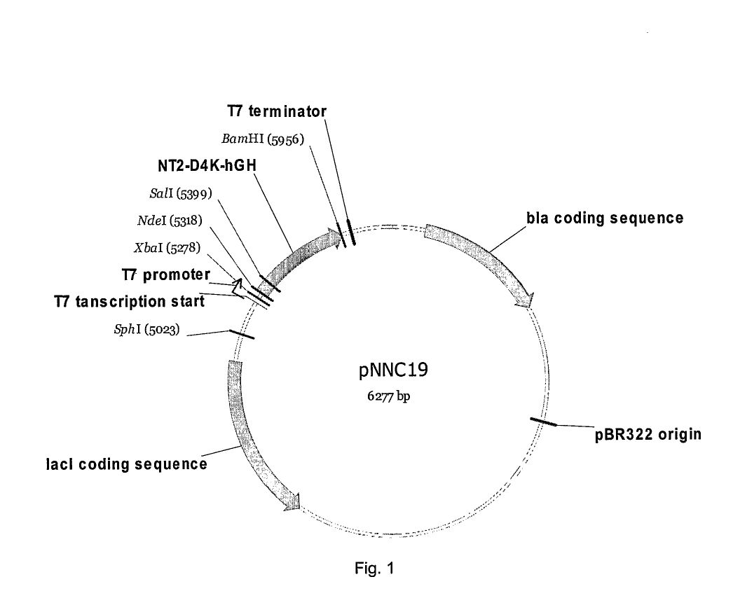

FIG. 2: Diagnostic PCR of selected clones. M shows 1 Kb marker. Lane 1 shows parental vector; lane 2 OGP-hGH; lane 3 OGP(1-9)-hGH; lane 4 OGP-OGP-hGH; and lane 5 OGP(1-9)-OGP(1-9)-hGH. The same primer set is used in all reactions and the expected size differences are reflected on the gel (OGP-hGH: 277 bp, OGP-OGP-hGH: 319 bp, OGP(1-9)-hGH: 262 bp, OGP(1-9)-OGP(1-9)-hGH: 289 bp). In addition, the primer set was unable to amplify the parental vector. Diagnostic primer set: OGP primer: 5′-tggctctgaaacgtcagggtcgta-3′, hGH Primer 5′-atgcggagcagctctaggftggat-3′.

FIG. 3: M shows molecular weight marker. Lane 1-4 shows BL-21 transformed with the OGP-hGH expression vector. 1 Un-induced bacteria; 2 after induction with IPTG; 3 supernatant; and 4 pellet fraction. Lane 5-8 shows BL-21 transformed with the OGP(1-9)-hGH expression vector. 5 Un-induced bacteria; 6 after induction with IPTG; 7 supernatant; and 8 pellet fraction. The recombinant expression of OGP-hGH and OGP(1-9)-hGH are marked with arrows. Both fusion-proteins migrate at the predicted molecular weight.

FIG. 4: Western blot of OGP constructs. Lane 1; Molecular weight marker, lane 2; hGH standard, lanes 3-5; OGP-hGH, lanes 6-8; OGP(1-9)-hGH, lanes 9-11; OGP-OGP-hGH, lanes 12-14; 2 OGP(1-9)-OGP(1-9)-hGH. For each of the constructs, the three lanes show the protein preparation before induction and after sonication in two different dilutions.

FIG. 5: SDS-PAGE of OGP-hGH. Lane 1; Molecular weight marker, lane 2; hGH standard, lane 3; OGP-hGH non-reduced sample, lane 4; OGP-hGH reduced sample.

FIG. 6: SDS-PAGE of OGP-OGP-hGH. Lanes 1-9 are reduced, lanes 10-14 are non-reduced. Lanes 1-3; hGH standard, lanes 4-5; solubilised inclusion bodies, lane 6; refolded OGP-OGP-hGH, lane 7; application SP Sepharose column, lanes 8-9; purified OGP-OGP-hGH, lane 10; application SP Sepharose column, lane 11; solubilised inclusion bodies, lanes 12-14; purified OGP-OGP-hGH.

FIG. 7: SDS-PAGE of OGP-OGP-OGP-hGH. Lane 1; Molecular weight marker, lane 2; hGH standard, lane 3; OGP-OGP-OGP-hGH reduced sample, lane 4; OGP-OGPOGP-hGH non-reduced sample.

FIG. 8. Surface plasmon resonance analysis of hGH (circles), OGP-hGH (squares), and OGP-OGP-hGH (diamonds) binding to immobilized α₂-macroglobulin. The concentration of injected protein was 1500 nM. Association and dissociation phases lasted 10 and 9 min, respectively

### DEFINITIONS

In the present context, the term “OGP fusion protein” is intended to indicate a protein formed by the fusion of a first protein, e.g. a therapeutic protein, to the C-terminal of a second protein which is OGP or a variant thereof. It is to be understood that said fusion may be direct in the sense that the C-terminal of OGP or the variant thereof is bound directly to the N- or C-terminal of the above first protein. The fusion may also be via a linker wherein said linker at one end is bonded to the C-terminal of OGP or a variant thereof, and at the other end is bonded to the above first protein. The point of attachment in the above first protein may at any of the amino acid residues constituting said protein, i.e. the C-terminal, the N-terminal or any of the amino acid residues in between. Unless otherwise stated, the term “fusion” is not intended to imply that that the fusion protein is produced by any particular method.

In the present context, a “linker” is a moiety which serves to connect the two parts of an OGP fusion protein, e.g., the OGP part and the above first protein part. In one embodiment said linker is a biradical selected from straight or branched C₁₋₅₀-alkylene, straight or branched C₂₋₅₀-alkenylene, straight or branched C₂₋₅₀-alkynylene, a 1 to 50-membered straight or branched chain comprising carbon and at least one N, O or S atom in the chain; C₃₋₈cycloalkylene; a 3 to 8-membered cyclic ring comprising carbon and at least one N, O or S atom in the ring; arylene; heteroarylene; or an amino acid biradical, the biradicals optionally being substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogen, C₁₋₆ alkoxy, C₁₋₆alkylthio, trifluoroalkoxy, alkoxycarbonyl, and haloalkyl. In another embodiment, said linker represents a polypeptide diradical comprising up to 50 amino acid residues, such as up to 40, 30, 20 or 10 amino acid residues. It may be desirable to cleave the OGP fusion protein at some point in which case a cleavage site, e.g. for enzymatic hydrolysis, may be comprised in the linker.

In the present context, the term “protein” is intended to indicate a sequence of amino acids bonded by peptide bonds. Preferably, a protein comprises more than 20 amino acid residues, wherein said amino acids may be codable or non-codable. It is to be understood that the term also is intended to include proteins which have been further derivatized, e.g. by the attachment of lipophilic or PEG groups, unless otherwise stated.

A “therapeutically effective amount” of a compound as used herein means an amount sufficient to cure, alleviate or partially arrest the clinical manifestations of a given disease and/or its complications. Effective amounts for each disease will depend e.g. on the severity of the disease or injury as well as the weight, sex, age and general state of the subject to be treated. It will be understood that determining an appropriate dosage may be achieved using routine experimentation, by constructing a matrix of values and testing, different points in the matrix, which is all within the ordinary skills of a trained physician or veterinary.

The term “treatment” and “treating” as used herein means the management and/or care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as, unless otherwise stated, to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs. It should be recognized that therapeutic regimens and prophylactic (preventative) regimens represent separate aspects of the invention.

The term “therapeutic protein” is intended to indicate a protein having one or more, therapeutic and/or biological activities in vivo (in an animal, commonly a chordate, and typically a mammal, such as a primate, for example a human). A therapeutic protein is useful to treat or ameliorate a disease, condition or disorder. Although the activity of therapeutic proteins ultimately is to be effected in vivo, there are many in vitro assays known to the person skilled in the art whereby therapeutic activity can be measured.

### DESCRIPTION OF THE INVENTION

The invention is partly based on the discovery that OGP or variants thereof when fused to proteins, such as e.g. therapeutic proteins extends the circulation time of said proteins in circulation. Without wishing to be bound by any specific theory, it is believed that OGP or the variant thereof in the fused protein retains the ability to bind to α₂M, and that this binding of the fused protein to α₂M makes the fused protein less susceptible to e.g. breakdown or renal clearance.

In one embodiment, the invention provides an OGP fusion protein comprising a first protein fused to the C-terminal of OGP or a variant thereof. The invention is intended to indude pharmaceutically acceptable salts of said OGP fusion proteins.

In one embodiment, the invention provides an OGP fusion protein containing a first protein fused to the C-terminal of OGP or a variant thereof.

In one embodiment, said first protein is a therapeutic protein. In a more particular aspect, the first protein is a therapeutic protein of mammalian (e.g., human) or synthetic origin/composition.

In one embodiment, said first protein comprises at least 30 amino acid residues.

In the present context, a “variant of OGP” is understood to be a variant which preferably binds to α₂M. By “binds to α₂M” is meant that the variant binds to α₂M to an extent whereby the circulation time of the fusion protein is increased compared to the circulation time of the protein which has been fused to the OGP variant. Binding may be quantified in terms of the dissociation constant, K_{D}, which is defined as

$${K}_{D} = \frac{[ {A}_{x} ] [ {A}_{y} ]}{[ {A}_{xy} ]}$$

wherein Aₓ, A_{y} and A_{xy} are the activities of “x”, “y” and “xy” in the system xy [symbol] x+y at 25° C. In one embodiment, the K_{D} for the OGP variant-α2M binding may be equal to that of the OGP-α2M binding. In another embodiment, the K_{D} for the OGP variant-α₂M binding is larger than that of the OGP-α₂M binding, such as up to 2 times, or such as up to 5 times, or such as up to 10 times, or such as up to 20 times, or such as up to 50 times, or such as up to 100 times larger. In another embodiment, the K_{D} for the OGP variant-α₂M binding is smaller than that of the OGP-α₂M binding, such as down to 90%, or such as down to 80%, or such as down to 70%, or such as down to 50%, or such as down to 20%, or such as down to 10%, or such as down to 1% of the K_{D} of the OGP-α₂M binding. In this context, the dissociation constant (Kd) may be determined as described in Yang et al. J Biol Chem 269, 18977-18984, 1994; Murai et al J Biol. Chem., 270, 19957-19993, 1995; Kawaura et al. Biosci. Biotechnol. Biochem., 67, 869-876, 2003. A binding to α₂M does not exclude a binding to other plasma proteins.

In one embodiment, the K_{D} for the OGP fusion protein-α₂M binding may be equal to that of the OGP-α₂M binding. In another embodiment, the K_{D} for the OGP fusion protein-α₂M binding is larger than that of the OGP-α₂M binding, such as up to 2 times, or such as up to 5 times, or such as up to 10 times, or such as up to 20 times, or such as up to 50 times, or such as up to 100 times larger. In another embodiment, the K_{D} for the OGP fusion protein-α₂M binding is smaller than that of the OGP-α₂M binding, such as down to 90%, or such as down to 80%, or such as down to 70%, or such as down to 50%, or such as down to 20%, or such as down to 10%, or such as down to 1% of the K_{D} of the OGP-α₂M binding.

In one embodiment, an OGP variant is obtained by adding, substituting, and/or deleting one or more amino acid residues from the OGP sequence. In particular, such substitutions typically are conservative in the sense that one amino acid residue is substituted by another amino acid residue from the same amino acid group, i.e. by another amino acid residue with similar physiochemical properties. Amino acid may conveniently be divided in the following groups based on their properties: Basic amino acids (such as arginine, lysine, histidine), acidic amino acids (such as glutamic acid and aspartic acid), polar amino acids (such as glutamine and asparagine), hydrophobic amino acids (such as leucine, isoleucine, valine), aromatic amino acids (such as phenylalanine, tryptophan, tyrosine) and small amino acids (such as glycine, alanine, serine, threonine, methionine). Amino acid residues in OGP may also be substituted with non-codable amino acid residues, and this also forms part of the present invention.

In one embodiment, up to 5 amino acids, such as 1, 2, 3, 4 or 5 amino acids have been substituted.

In one embodiment, the variant is formed by deleting up to 5, such as 1, 2, 3, 4 or 5 amino acid residues from the C-terminal of OGP. In particular, said variant is OGP(1-9).

In one embodiment, a linker made of up to 30 amino acid residues is inserted between the first protein and OGP or the variant thereof. In particular, this linker may be made of up to 25 amino acid residues, such as up to 20 amino acid residues, such as up to 15 amino acid residues, such as up to 10 amino acid residues, such as up to 5 amino acid residues, such as 1, 2, 3 or 4 amino acid residues. Particular mentioning is made of OGP, OGP-OGP, OGP(1-9) and OGP(1-9)-OGP(1-9) as linkers.

OGP comprises several charged residues, and the fusion of OGP or a variant thereof to a first protein is thus likely to change the pl of the fused protein compared to that of the first protein. As pl is a determining factor for the pH-solubility profile of proteins, the fusion protein may have a different pH-solubility profile than the first protein. If the resulting pH-solubility profile is unfavorable with respect to a particular application, the pi may be altered by careful selection of the linker. By selection of a suitably charged linker, the pi of the fusion protein may be altered into a useful range, e.g. remained unchanged relative to the first protein. In one embodiment, the invention therefore provides a fusion protein comprising a linker, wherein said fusion protein has an altered solubility, such as an increased solubility, compared to a reference protein, which reference protein is the fusion protein without said linker.

In one embodiment, said linker is selected so as to minimize any immunological response provoked by the fusion protein. It is believed that this may be achieved by using protein sequences already present, e.g. in the human body. Albumin is one example of such a protein, and in particular the sequence consisting of amino acid numbers 295-304 thereof (NDEMPADLPS (SEQ ID NO:34)), which comprises many charged residues, is useful a linker.

In one embodiment, the present invention relates to OGP variants as indicated above.

In one embodiment, the invention provides a method of increasing the circulation time of a protein, the method comprising fusing said protein to the C-terminal of OGP or a variant thereof. In one embodiment, said first protein is not salmon calcotonin.

An increase in circulation time may be quantified as a decrease in clearance (CL) or as an increase in mean residence time (MRT). Fusion proteins of the present invention for which the CL is decreased to below 75%, such as 50% or less of the CL of the protein to which OGP or a variant thereof has been fused is said to have an increased circulation time. Fusion proteins of the present invention for which MRT is increased to above 120%, such as 150% or more of the MRT of the protein to which OGP or a variant thereof has been fused is said to have an increased circulation time. Clearance and mean residence time can be assessed in standard pharmacokinetic studies using suitable test animals, such as e.g. normal, Sprague-Dawley male rats, mice or cynomolgus monkeys. Typically the mice and rats are in injected in a single subcutaneous bolus, while monkeys may be injected in a single subcutaneous bolus or in a single iv dose. The amount injected depends on the test animal. Subsequently, blood samples are taken over a period of one to five days as appropriate for the assessment of CL and MRT. The blood samples are conveniently analysed by ELISA techniques.

In one embodiment, the invention provides fusion proteins selected from

| | |
|---|---|
| OGP-hGH,; | (SEQ ID NO:1) |
| OGP(1-9)-hGH,; | (SEQ ID NO:2) |
| OGP-OGP-hGH,; | (SEQ ID NO:3) |
| OGP(1-9)-OGP(1-9)-hGH; | (SEQ ID NO:4) |
| OGP(1-9)-OGP(1-9)-OGP(1-9)-hGH; | (SEQ ID NO:17) |
| OGP-OGP-OGP-hGH; | (SEQ ID NO:18) |
| OGP-OGP-NDEMPADLPS-hGH; | (SEQ ID NO:19) |
| and | |
| OGP(1-9)-OGP(1-9)-NDEMPADLPS-hGH | (SEQ ID NO:20) |

wherein hGH denotes human growth hormone.

In one embodiment, the invention provides nucleic acid constructs encoding proteins of the present invention.

The fusion proteins of the present invention may be prepared in a number of different ways. They may be synthesized using protein synthetic methods well-known to persons skilled in the art. It is also possible to express the protein to be fused with OGP or a variant thereof and the OGP or OGP variant separately in suitable hosts and fuse the two proteins subsequently. In a particular embodiment, however, the OGP fusion protein is expressed as such in a suitable host after incorporation of a suitable nucleic acid construct into said host.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a protein of interest. The construct may optionally contain other nucleic acid segments.

The nucleic acid construct of the invention encoding the protein of the invention may suitably be of genomic or cDNA origin, for instance obtained by preparing a genomic or cDNA library and screening for DNA sequences coding for all or part of the protein by hybridization using synthetic oligonucleotide probes in accordance with standard techniques (cf. Sambrook et al., supra). For the present purpose, the DNA sequence encoding the protein is preferably of human origin, i.e. derived from a human genomic DNA or cDNA library. In particular, the DNA sequence may be of human origin, e.g. cDNA from a particular human organ or cell type or a gene derived from human genomic DNA.

The nucleic acid construct of the invention encoding the protein may also be pre-pared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239 (1988), 487-491.

The nucleic acid construct is preferably a DNA construct which term will be used exclusively in the following.

Recombinant Vector

In a further aspect, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the protein of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the protein.

The promoter may be any DNA sequence which shows transcriptional activity in the host cell of choice and may be derived from genes encoding proteins either homologous or heterologous to the host cell.

Examples of suitable promoters for directing the transcription of the DNA encoding the protein of the invention in mammalian cells are the SV40 promoter (Subramani et al., Mol. Cell. Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39 K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4-c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters.

Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gen, or the Bacillus pumilus xylosidase gene, or by the phage Lambda P_{R} or P_{L} promoters or the E. coli lac, trp or tac promoters.

The DNA sequence encoding the protein of the invention may also, if necessary, be operably connected to a suitable terminator, such as the human growth hormone terminator (Palmiter et al., op cit.) or (for fungal hosts) the TPI1 (Alber and Kawasaki, op. cit.) or ADH3 (McKnight et al., op cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector of the invention may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

When the host cell is a bacterial cell, sequences enabling the vector to replicate are DNA polymerase III complex encoding genes and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pvrG, arqB, niaD and sC.

To direct a protein of the present invention into the secretory pathway of the host cells, a secretory signal sequence (also known as a leader sequence, prepro sequence or pre sequence) may be provided in the recombinant vector. The secretory signal sequence is joined to the DNA sequence encoding the protein in the correct reading frame. Secretory signal sequences are commonly positioned 5′ to the DNA sequence encoding the protein. The secretory signal sequence may be that normally associated with the protein or may be from a gene encoding another secreted protein.

For secretion from yeast cells, the secretory signal sequence may encode any signal peptide which ensures efficient direction of the expressed protein into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and uptream of the DNA sequence encoding the protein. The function of the leader peptide is to allow the expressed protein to be directed from the endoplasmic reticulum to the Golgi apparatus and further to a secretory vesicle for secretion into the culture medium (i.e. exportation of the protein across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the present protein, the promoter and optionally the terminator and/or secretory signal sequence, respectively, and to insert them into suitable vectors containing the information necessary for replication, are well known to persons skilled in the art (cf., for instance, Sambrook et al., op.cit.).

Host Cells

The host cell into which the DNA construct or the recombinant vector of the invention is introduced may be any cell which is capable of producing the present protein and includes bacteria, yeast, fungi and higher eukaryotic cells.

Examples of bacterial host cells which, on cultivation, are capable of producing the protein of the invention are grampositive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gramnegative bacteria such as Echerichia coli. The transformation of the bacteria may be effected by protoplast transformation or by using competent cells in a manner known per se (cf. Sambrook et al., supra).

When expressing the protein in bacteria such as E. coli, the protein may be retained in the cytoplasm, typically as insoluble granules (known as inclusion bodies), or may be directed to the periplasmic space by a bacterial secretion sequence. In the former case, the cells are lysed and the granules are recovered and denatured after which the protein is refolded by diluting the denaturing agent. In the latter case, the protein may be recovered from the periplasmic space by disrupting the cells, e.g. by sonication or osmotic shock, to release the contents of the periplasmic space and recovering the protein.

Examples of suitable mammalian cell lines are the COS (ATCC CRL 1650), BHK (ATCC CRL 1632, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous proteins therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding the protein of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al., 1989, Gene 78: 147-156.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous proteins therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidopteracell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting the expression of the present protein, after which the resulting protein is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The protein produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of protein in question.

Transgenic Animals

It is also within the scope of the present invention to employ transgenic animal technology to produce the present protein. A transgenic animal is one in whose genome a heterologous DNA sequence has been introduced. In particular, the protein of the invention may) be expressed in the mammary glands of a non-human female mammal, in particular one which is known to produce large quantities of milk. Examples of preferred mammals are livestock animals such as goats, sheep and cattle, although smaller mammals such as mice, rabbits or rats may also be employed.

The DNA sequence encoding the present protein may be introduced into the animal by any one of the methods previously described for the purpose. For instance, to obtain expression in a mammary gland, a transcription promoter from a milk protein gene is used. Milk protein genes include the genes encoding casein (cf. U.S. Pat. No. 5,304,489), beta-lactoglobulin, alpha-lactalbumin and whey acidic protein. The currently preferred promoter is the beta-lactoglobulin promoter (cf. Whitelaw et al., Biochem J. 286, 1992, pp. 31-39).

It is generally recognized in the art that DNA sequences lacking introns are poorly expressed in transgenic animals in comparison with those containing introns (cf. Brinster et al., Proc. Natl. Acad. Sci. USA 85, 1988, pp. 836-840; Palmiter et al., Proc. Natl. Acad. Sci. USA 88, 1991, pp. 478-482; Whitelaw et al., Transgenic Res. 1, 1991, pp. 3-13; WO 89/01343; WO 91/02318). For expression in transgenic animals, it is therefore preferred, whenever possible, to use genomic sequences containing all or some of the native introns of the gene encoding the protein of interest. It may also be preferred to include at least some introns from, e.g. the beta-lactoglobulin gene. One such region is a DNA segment which provides for intron splicing and RNA polyadenylation from the 3′ non-coding region of the ovine beta-lactogloblin gene. When substituted for the native 3′ non-coding sequences of a gene, this segment may will enhance and stabilise expression levels of the protein of interest. It Pmay also be possible to replace the region surrounding the initiation codon of the protein of interest with corresponding sequences of a milk protein gene. Such replacement provides a putative tissue-specific initiation environment to enhance expression.

For expression of the present protein in transgenic animals, a nucleotide sequence encoding the protein is operably linked to additional DNA sequences required for its expression to produce expression units. Such additional sequences include a promoter as indicated above, as well as sequences providing for termination of transcription and polyadenylation of mRNA. The expression unit further includes a DNA sequence encoding a secretory signal sequence operably linked to the sequence encoding the protein. The secretory signal sequence may be one native to the protein or may be that of another protein such as a milk protein (cf. von Heijne et al., Nucl. Acids Res. 14, 1986, pp. 4683-4690; and U.S. Pat. No. 4,873,316).

Construction of the expression unit for use in transgenic animals may conveniently be done by inserting a DNA sequence encoding the present protein into a vector containing the additional DNA sequences, although the expression unit may be constructed by essentially any sequence of ligations. It is particularly convenient to provide a vector containing a DNA sequence encoding a milk protein and to replace the coding region for the milk protein with a DNA sequence coding for the present protein, thereby creating a fusion which includes expression control sequences of the milk protein gene.

The expression unit is then introduced into fertilized ova or early-stage embryos of the selected host species. Introduction of heterologous DNA may be carried out in a number of ways, including microinjection (cf. U.S. Pat. No. 4,873,191), retroviral infection (cf. Jaenisch, Science 240, 1988, pp. 1468-1474) or site-directed integration using embryonic stem cells (reviewed by Bradley et al., Bio/Technology 10, 1992, pp. 534-539). The ova are then implanted into the oviducts or uteri of pseudopregnant females and allowed to develop to term. Offspring carrying the introduced DNA in their germ line can pass the DNA on to their progeny, allowing the development of transgenic herds.

General procedures for producing transgenic animals are known in the art, cf. for instance, Hogan et al., Manipulating the Mouse Embryo: A Laboratory Manual, Cold Spring Harbor Laboratory, 1986; Simons et al., Bio/Technology 6, 1988, pp. 179-183; Wall et al., Biol. Reprod. 32, 1985, pp. 645-651; Buhler et al., Bio/Technology 8, 1990, pp. 140-143; Ebert et al., Bio/Technology 6: 179-183, 1988; Krimpenfort et al., Bio/Technology 9: 844-847, 1991, Wall et al., J. Cell. Biochem. 49:113-120, 1992; U.S. Pat. No. 4,873,191, U.S. Pat. No. 4,873,316; WO 88/00239, WO 90/05188; WO 92/11757 and GB 87/00458. Techniques for introducing heterologous DNA sequences into mammals and their germ cells were originally developed in the mouse. See, e.g. Gordon et al., Proc. Natl. Acad. Sci. USA 77: 7380-7384, 1980, Gordon and Ruddle, Science 214: 1244-1246, 1981; Palmiter and Brinster, Cell 41: 343-345, 1985; Brinster et al., Proc. Natl. Acad. Sci. USA 82: 4438-4442, 1985; and Hogan et al. (ibid.). These techniques were subsequently adapted for use with larger animals, including livestock species (see e.g., WO 88/00239, WO 90/01588 and WO 92/11757; and Simons et al., Bio/Technology 6: 179-183, 1988). To summarize, in the most efficient route used to date in the generation of transgenic mice or livestock, several hundred linear molecules of the DNA of interest are injected into one of the pro-nuclei of a fertilized egg according to techniques which have become standard in the art. Injection of DNA into the cytoplasm of a zygote can also be employed.

In another embodiment, the protein to be fused with OGP or a variant thereof and OGP or the variant thereof are expressed separately. The above protein may then be reacted with a bi-functional linker (activation) whereby the linker is bonded to the protein via a first functional groups. The activated protein is subsequently reacted with OGP or a variant thereof whereby OGP or the variant thereof is bonded to the linker via the second functional group. It is clear to the person skilled in the art that the reaction order may be reversed so that the linker is first reacted with OGP or a variant thereof followed by a reaction with the protein to be fused.

Numerous functional groups have been disclosed in the literature which are useful for forming a bond between a protein and a linker. Relevant references are, e.g. WO 03/044056 and Biomaterials, 22, 405-417, 2001. Typically, groups in proteins which are useful points of attachments are amines, hydroxyls, thiols, aldehydes and ketones, which may be present in the native protein or which may be generated, e.g. by oxidation. When the protein to be fused to OGP or a variant thereof and OGP or the variant is fused via a linker, the linker may in principle be attached to any amino acid residue in the protein to be fused to OGP or a variant thereof.

The protein to be fused to OGP or a variant thereof may be further derivatised, e.g. by attachment of lipophilic or PEG groups to further modify the properties. Also, upon fusion to OGP or a variant thereof, the resulting fused protein may be further derivatized, e.g. by attachment of lipophilic or PEG groups to further modify the properties of the fused protein. Any protein may in principle be fused to OGP or a OGP variant according to the methods of the invention. Such proteins include enzymes, peptide hormones, growth factors, antibodies, cytokines, receptors, lymphokines, and vaccines antigens, and particular mentioning is made of therapeutic proteins, such as insulin, glucagons-like peptide 1 (GLP-1), glucagons-like peptide 2 (GLP-2), growth hormone, cytokines, trefoil factor peptides (TTF), peptide melanocortin receptor modifiers, IL-20, IL-21, IL-28a, IL-29, IL-31 and Factor VII compounds. In one embodiment, the invention relates to OGP fusion proteins which subsequent to the fusion of the OGP or OGP variant is further derivatized, e.g. with lipophilic groups to obtain a further modification of the properties of the protein.

Particular applicable insulin is human insulin. In the present context the term “human insulin” refers to naturally produced insulin or recombinantly produced insulin. Recombinant human insulin may be produced in any suitable host cell, for example the host cells may be bacterial, fungal (including yeast), insect, animal or plant cells. Many insulin compounds have been disclosed in the literature, and they too are particular useful in the methods of the pre-sent invention. By “insulin compound” (and related expressions) is meant human insulin in which one or more amino acids have been deleted and/or replaced by other amino acids, including non-codeable amino acids, and/or human insulin comprising additional amino acids, i.e. more than 51 amino acids, and/or human insulin in which at least one organic substituent is bound to one or more of the amino acids.

Examples of GLP-1 applicable in the methods of the present invention include human GLP-1 and GLP-1 compounds. Human GLP-1 is a 37 amino acid residue peptide originating from preproglucagon which is synthesised i.a. in the L-cells in the distal ileum, in the pancreas and in the brain. GLP-1 is an important gut hormone with regulatory function in glucose metabolism and gastrointestinal secretion and metabolism. Processing of preproglucagon to give GLP-1 (7-36)-amide, GLP-1 (7-37) and GLP-2 occurs mainly in the L-cells. The fragments GLP-1 (7-36)-amide and GLP-1 (7-37) are both glucose-dependent insulinotropic agents. In the past decades a number of structural analogues of GLP-1 were isolated from the venom of the Gila monster lizards (Heloderma suspectum and Heloderma horridum). Exendin-4 is a 39 amino acid residue peptide isolated from the venom of Heloderma horridum, and this peptide shares 52% homology with GLP-1. Exendin-4 is a potent GLP-1 receptor agonist which has been shown to stimulate insulin release and ensuring lowering of the blood glucose level when injected into dogs. The group of GLP-1(7-37) and exendin-4(1-39) and certain fragments, analogues and derivatives thereof (designated GLP-1 compounds herein) are potent insulinotropic agents, and they are all applicable in the method of the pre-sent invention. Insulinotropic fragments of GLP-1 (1-37) are insulinotropic peptides for which the entire sequence can be found in the sequence of GLP-1 (1-37) and where at least one terminal amino acid has been deleted. Examples of insulinotropic fragments of GLP-1 (1-37) are GLP-1 (7-37) wherein the amino acid residues in positions 1-6 of GLP-1 (1-37) have been deleted, and GLP-1 (7-36) where the amino acid residues in position 1-6 and 37 of GLP-1 (1-37) have been deleted. Examples of insulinotropic fragments of exendin-4(1-39) are exendin-4(1-38) and exendin-4(1-31). The insulinotropic property of a compound may be determined by in vivo or in vitro assays well known in the art. For instance, the compound may be administered to an animal and monitoring the insulin concentration over time. Insulinotropic analogs of GLP-1 (1-37) and exendin-4(1-39) refer to the respective molecules wherein one or more of the amino acids residues have been exchanged with other amino acid residues and/or from which one or more amino acid residues have been deleted and/or from which one or more amino acid residues have been added with the proviso that said analogue either is insulinotropic or is a prodrug of an insulinotropic compound.

GLP-2 and GLP-2 compounds may also be modified by the methods provided by the present invention. In the present context a GLP-2 compound binds to a GLP-2 receptor, preferably with an affinity constant (K_{D}) or a potency (EC₅₀) of below 1 μM, e.g. below 100 nM. The term “GLP-2 compound” is intended to indicate human GLP-2 in which one or more amino acid residue has been deleted and/or replaced by another amino acid residue, natural or unnatural, and/or human GLP-2 comprising additional amino acid residues, and/or human GLP-2 in which at least one organic substituent is bound to one or more of the amino acid residues. In particular, those peptides are considered, which amino acid sequence exhibit at any sequence of 33 consecutive amino acids more than 60% of the amino acid sequence of human GLP-2. Also those peptides are considered, which amino acid sequence exhibit at any sequence of 37 consecutive amino acids more than 60% of the amino acid sequence of human GLP-2 when up to four amino acids are deleted from the amino acid sequence. Also those peptides are considered, which amino acid sequence exhibit at any sequence of 31 consecutive amino acids more than 60% of the amino acid sequence of GLP-2, when up to two amino acids are added to their amino acid sequence. The term “GLP compounds” also includes natural allelic variations that may exist and occur from one individual to another. Also, degree and location of glycosylation or other post-translation modifications may vary depending on the chosen host cells and the nature of the host cellular environment. Candidate GLP-2 compounds, which may be used according to the present invention include the GLP-2 compounds described in WO 96/32414, WO 97/39031, WO 98/03547, WO 96/29342, WO 97/31943, WO 98/08872, which are all incorporated herein by reference.

Factor VII compounds applicable in the methods of the present invention encompasses wild-type Factor VII (i.e., a polypeptide having the amino acid sequence disclosed in U.S. Pat. No. 4,784,950), as well as variants of Factor VII exhibiting substantially the same or improved biological activity relative to wild-type Factor VII, Factor VII-related polypeptides as well as Factor VII derivatives and Factor VII conjugates. The term “Factor VII compounds” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. Such variants of Factor VII may exhibit different properties relative to human Factor VII, including stability, phospholipid binding, altered specific activity, and the like.

As used herein, “Factor VII-related polypeptides” encompasses polypeptides, including variants, in which the Factor VIIa biological activity has been substantially modified or reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The term “Factor VII derivative” as used herein, is intended to designate wild-type Factor VII, variants of Factor VII exhibiting substantially the same or improved biological activity relative to wild-type Factor VII and Factor VII-related polypeptides, in which one or more of the amino acids of the parent peptide have been chemically modified, e.g. by alkylation, PEGylation, acylation, ester formation or amide formation or the like. This includes but are not limited to PEGylated human Factor VIIa, cysteine-PEGylated human Factor VIIa and variants thereof.

The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

The biological activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively). For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa to produce of Factor Xa in a system comprising TF embedded in a lipid membrane and Factor X. (Persson et al., J. Biol. Chem. 272: 19919-19924, 1997); (ii) measuring Factor X hydrolysis in an aqueous system; (iii) measuring its physical binding to TF using an instrument based on surface plasmon resonance (Persson, FEBS Letts. 413: 359-363, 1997) and (iv) measuring hydrolysis of a synthetic substrate.

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay, proteolysis assay, or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay, proteolysis assay, or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids.

The terms “variant” or “variants”, as used herein, is intended to designate Factor VII having the sequence of wild-type factor VII, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. The “variant” or “variants” within this definition still have FVII activity in its activated form. In one embodiment a variant is 70% identical with the sequence of wild-type Factor VII. In one embodiment a variant is 80% identical with the sequence of wild-type factor VII. In another embodiment a variant is 90% identical with the sequence of wild-type factor VII. In a further embodiment a variant is 95% identical with the sequence of wild-type factor VII.

Growth hormone applicable in the methods of the present invention includes human growth hormone (hGH), which sequence and characteristics are set forth in, e.g. Hormone Drugs, Gueriguian, U.S.P. Covention, Rockvill, 1982 and growth hormone compounds. The term “growth hormone compound” is intended to indicate human growth hormone (hGH) in which one or more amino acid residues have been deleted and/or replaced by other amino acid residues, natural or unnatural, and/or hGH comprising addition amino acid residues, natural or unnatural, and/or hGH in which at least one organic substituent is bound to one or more organic substituent. Particular mentioning is made of the 191 native amino acid sequence (somatropin) and the 192 amino acid N-terminal methionine species (somatrem). Examples of cytokines which could be modified using the method of the present invention include erythropoietin (EPO), thrombopoietin, INF-α, IFN-β, IFN-γ, TNF-α, interleukin-1β (IL-1-β), IL-3, IL-4, IL-5, IL-10, IL-12, IL-15, IL-18, IL-19, IL-20, IL-21 IL-24, IL-28a, IL-29, IL-31, grannolyte colony-stimulating factor (G-CSF), GM-CSF, and chemokines such as machrophage inflammatory protein-1 (MIP-1) gamma interferon inducible protein and monokines induced by IFNγ (MIG).

Particular examples of IL-19 applicable in the methods of the present invention include those disclosed WO 98/08870 (Human Genome Science), which is incorporated herein by reference.

Particular examples of applicable IL-20 include those disclosed in WO 99/27103 (Zymogenetics), which is incorporated herein by reference. In the present context, IL-20 is intended to indicate IL-20 itself and fragments thereof as well as polypeptides being at least 90% identical to IL-20 or fragments thereof.

Examples of IL-21 applicable in the methods of the present invention include those disclosed in WO 00/53761 (Zymogenetics), which is incorporated herein by reference.

Examples of IL-28a applicable in the methods of the present invention include those disclosed in WO 02/92762 and WO 02/86087, both of which are incorporated herein by reference.

Examples of IL-29 applicable in the methods of the present invention include those disclosed in WO 02/02627 and WO 02/092762, both of which are incorporated herein by reference.

Examples of IL-31 applicable in the methods of the present invention include those disclosed in WO Feb. 3, 20060090, which is incorporated herein by reference.

TTF are applicable in the methods of the present invention. TTF peptides are a family of peptides found mainly in association with the gastrointestinal tract. Particular mentioning is made of breast cancer associated pS2 peptide (TFF-1), which is known from human, mouse, and rat, spasmolytical polypeptide (TFF-2), which is known from human, pig, rat, and mouse and intestinal trefoil factor (TFF-3), known from human, rat and mouse.

Other peptides from the TFF family applicable in the methods of the present invention include those disclosed in WO 02/46226 (Novo Nordisk), which is included herein by reference. Other peptides of the TFF family include TFF-1 and TFF-3 dimers as those disclosed in WO 96/06861 (Novo Nordisk), which is incorporated herein by reference.

Several melanorcortin receptors are known, and particular mentioning of peptides applicable for the methods of the present invention is made of peptidic melanocortin-4 receptor agonists, which are known to have an appetite suppressive effect. Particular mentioning is made of peptides or proteins disclosed in the following patent documents, which are all incorporated herein by reference: U.S. Pat. No. 6,054,556 (Hruby), WO 00/05263 (William Harvey Research), WO 00/35952 (Melacure), WO 00/35952 (Melacure), WO 00/58361 (Procter & Gamble), WO 01/52880 (Merck), WO 02/26774 (Procter & Gamble), WO 03/06620 (Palatin), WO 98/27113 (Rudolf Magnus Institute) and WO 99/21571 (Trega).

Other classes of peptides or proteins which are applicable in the methods of the present invention include enzymes. Many enzymes are used for various industrial purposes, and particular mentioning is made of hydrolases (proteases, lipases, cellulases, esterases), oxidoreductases (laccases, peroxidaxes, catalases, superoxide dismutases, lipoxygenases), transferases and isomerases.

Other peptides or proteins applicable in the methods of the present invention include ACTH, corticotropin-releasing factor, angiotensin, calcitonin, glucagon, IGF-1, IGF-2, enterogastrin, gastrin, tetragastrin, pentagastrin, urogastrin, epidermal growth factor, secretin, nerve growth factor, thyrotropin releasing hormone, somatostatin, growth hormone releasing hormone, somatomedin, parathyroid hormone, thrombopoietin, erythropoietin, hypothalamic releasing factors, prolactin, thyroid stimulating hormones, endorphins, enkephalins, vasopressin, oxytocin, opiods and analogues thereof, asparaginase, arginase, arginine deaminase, adenosine deaminase and ribonuclease.

Insulin is used to treat or prevent diabetes, and in one embodiment, the present invention thus provides a method of treating type 1 or type 2 diabetes, the method comprising administering to a subject in need thereof a therapeutically effective amount of an OGP fusion protein comprising insulin or an insulin compound according to the present invention.

In another embodiment, the invention provides the use of an OGP fusion protein comprising insulin or an insulin compound according to the present invention in the manufacture of a medicament used in the treatment of type 1 or type 2 diabetes.

GLP-1 may be used in the treatment of hyperglycemia, type 2 diabetes, impaired glucose tolerance, type 1 diabetes, obesity, hypertension, syndrome X, dyslipidemia, β-cell apoptosis, β-cell deficiency, inflammatory bowel syndrome, dyspepsia, cognitive disorders, e.g. cognitive enhancing, neuroprotection, atheroschlerosis, coronary heart disease and other cardiovascular disorders. In one embodiment, the present invention thus provides a method of treating said diseases, the method comprising administering to a subject in need thereof a therapeutically effective amount of an OGP fusion protein comprising GLP-1 or a GLP-1 compound according to the present invention.

In another embodiment, the invention provides the use of an OGP fusion protein comprising GLP-1 or a GLP-1 compound according to the present invention in the manufacture of a medicament used in the treatment of the above mentioned diseases.

GLP-2 may be used in the treatment of intestinal failure leading to malabsorption of nutrients in the intestines, and in particular GLP-2 may be used in the treatment of small bowel syndrome, Inflammatory bowel syndrome, Crohn's disease, colitis including collagen colitis, radiation colitis, post radiation atrophy, non-tropical (gluten intolerance) and tropical sprue, damaged tissue after vascular obstruction or trauma, tourist diarrhea, dehydration, bacteremia, sepsis, anorexia nervosa, damaged tissue after chemotherapy, premature infants, schleroderma, gastritis including atrophic gastritis, postantrectomy atrophic gastritis and helicobacter pylori gastritis, ulcers, enteritis, cul-de-sac, lymphatic obstruction, vascular disease and graft-versus-host, healing after surgical procedures, post radiation atrophy and chemotherapy, and osteoporosis. It is therefore an intension of the present invention to provide methods of treating the above diseases, the method comprising administering to a subject in need thereof a therapeutically effective amount of an OGP fusion protein comprising GLP-2 or a GLP-2 compound according to this invention.

In another embodiment, the present invention provides the use of an OGP fusion protein comprising GLP-2 or a GLP-2 conjugate according to this invention in the manufacture of a medicament used in the treatment of the above mentioned diseases.

Growth hormone may be used in the treatment of diseases or states which will benefit from an increase in the amount of circulating growth hormone. In particular, the invention provides a method for the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucucorticoid treatment inchildren, the method comprising administering to a patient in need thereof a therapeutically effective amount of an OGP fusion protein comprising growth hormone or a growth hormone compound according to the present invention.

In one aspect, the invention provides a method for the acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of an OGP fusion protein comprising growth hormone or a growth hormone compound according to the present invention.

In one embodiment, the invention relates to the use of an OGP fusion protein comprising growth hormone or a growth hormone compound according to the present invention in the manufacture of medicament for the treatment of diseases benefiting from an increase in the growth hormone plasma level, such as the diseases mentioned above.

Cytokines are implicated in the etiology of a host of diseases involving the immune system. In particular it is mentioned that IL-20 could be involved in psoriasis and its treatment, and 1-21 is believed to be involved in cancer and could constitute a treatment to this disease. In one embodiment, the invention provides a method for the treatment of psoriasis comprising the administration of an OGP fusion protein comprising, such as e.g. containing an IL-20 conjugate according to the present invention. In another embodiment, the invention relates to the use of an OGP fusion protein comprising an IL-20 conjugate of the present invention in the manufacture of a medicament used in the treatment of psoriasis.

In another embodiment, the present invention relates to a method of treating cancer, the method comprising administration of an OGP fusion protein comprising an IL-21 conjugate of the present invention to a subject in need thereof.

In another embodiment, the invention relates to the use of an OGP fusion protein comprising an IL-21 conjugate according to the present invention in the manufacture of a medicament used in the treatment of cancer.

TTF peptides may be used to increase the viscosity of muscus layers in subject, to reduce secretion of salvia, e.g. where the increase salvia secretion is caused by irradiation therapy, treatment with anticholinergics or Sjögren's syndrome, to treat allergic rhinitis, stress induced gastric ulcers secondary to trauma, shock, large operations, renal or liver diseases, treatment with NSAID, e.g. aspirin, steroids or alcohol. TTF peptides may also be used to treat Chrohn's disease, ulcerative colitis, keratoconjunctivitis, chronic bladder infections, intestinal cystitis, papillomas and bladder cancer. In one embodiment, the invention thus relates the a method of treating the above mention diseases or states, the method comprising administering to a subject patient in need thereof a therapeutically effective amount of OGP fusion protein comprising TTF according to the present invention.

In another embodiment, the invention relates the use of OGP fusion protein comprising TTF of the present invention in the manufacture of a medicament for the treatment of the above mentioned diseases or states.

Melanocortin receptor modifiers, and in particular melanorcortin 4 receptor agonists have been implicated the treatment and prevention of obesity and related diseases. In one embodiment, the present invention provides a method for preventing or delaying the progression of impaired glucose tolerance (IGT) to non-insulin requiring type 2 diabetes, for pre-venting or delaying the progression of non-insulin requiring type 2 diabetes to insulinj requiring diabetes, for treating obesity and for regulating the appetite. Melanocortin 4 receptor agonists have also been implicated in the treatment of diseases selected from atherosclerosis, hypertension, diabetes, type 2 diabetes, impaired glucose tolerance (IGT), dyslipidemia, coronary heart disease, gallbladder disease, gall stone, osteoarthritis, cancer, sexual dysfunction and the risk of premature death. In one embodiment, the invention thus provides a method of treating the above diseases or states, the method comprising administering to a subject in need thereof a therapeutically effective amount of an OGP fusion protein comprising a melanocortin 4 receptor agonist of the present invention.

In still another embodiment, the invention relates to the use of an OGP fusion protein comprising a melanocortin 4 receptor agonist of the present invention in the manufacture of a medicament for the treatment of the above mentioned diseases or states.

Factor VII compounds have been implicated in the treatment of disease related to coagulation, and biological active Factor VII compounds in particular have been implicated in the treatment of hemophiliacs, hemophiliacs with inhibitors to Factor VII and IX, patients with thrombocytopenia, patients with thrombocytopathies, such as Glanzmann's thrombastenia platelet release defect and storage pool defects, patient with von Willebrand's disease, patients with liver disease and bleeding problems associated with traumas or surgery. Biologically inactive Factor VII compounds have been implicated in the treatment of patients being in hypercoagluable states, such as patients with sepsis, deep-vein thrombosis, patients in risk of myocardial infections or thrombotic stroke, pulmonary embolism, patients with acute coronary syndromes, patients undergoing coronary cardiac, prevention of cardiac events and restenosis for patient receiving angioplasty, patient with peripheral vascular diseases, and acute respiratory distress syndrome. In one embodiment, the invention thus provides a method for the treatment of the above mentioned diseases or states, the method comprising administering to a subject in need thereof a therapeutically effective amount of a an OGP fusion protein comprising a Factor VII compound according to the present invention.

In another embodiment, the invention provides the use of an OGP fusion protein comprising a Factor VII compound according to the present invention in the manufacture of a medicament used in the treatment of the above mentioned diseases or states.

Many diseases are treated using more than one medicament in the treatment, either concomitantly administered or sequentially administered. It is therefore within the scope of the present invention to use the peptide conjugates of the present invention in therapeutic methods for the treatment of one of the above mentioned diseases in combination with one or more other therapeutically active compound normally used to in the treatment said disease. By analogy, it is also within the scope of the present invention to use the peptide conjugates of the present invention in combination with other therapeutically active compounds normally used in the treatment of one of the above mentioned diseases in the manufacture of a medicament for said disease.

The above therapeutic methods may comprising administration via any suitable route, such as the oral, rectal, nasal, pulmonary, topical (including buccal, sublingual), transdermal, intracisternal, intraperitoneal, vaginal, parenteral (including subcutaneous, intramuscular, intrathecal, intravenous and intradermal) route, the parenteral route being preferred.

A typical parenteral dose is in the range of 10⁻⁹ mg/kg to about 100 mg/kg body weight per administration. Typical administration doses are from about 0.0000001 to about 10 mg/kg body weight per administration. The exact dose will depend on e.g. the activity of the compound, frequency and mode of administration, the sex, age and general condition of the subject to be treated, the nature and the severity of the disease or condition to be treated, the desired effect of the treatment and other factors evident to the person skilled in the art.

Typical dosing frequencies are twice daily, once daily, bi-daily, twice weekly, once weekly or with even longer dosing intervals. Due to the prolonged half-lifes of the fusion proteins of the present invention, a dosing regime with long dosing intervals, such as twice weekly, once weekly or with even longer dosing intervals is a particular embodiment of the invention.

Pharmaceutical Compositions

Another object of the present invention is to provide a pharmaceutical formulation comprising an OGP fusion protein compound which is present in a concentration from 10⁻¹⁵ mg/ml to 200 mg/ml, such as 10⁻¹⁰ mg/ml-5 mg/ml, and wherein said formulation has a pH from 2.0 to 10.0. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of an OGP fusion protein, and a buffer, wherein said OGP protein is present in a concentration from 0.1-100 mg/ml, and wherein said formulation has a pH from about 2.0 to about 10.0.

In a another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C₄-C₈ hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In a further embodiment of the invention the formulation further comprises a stabilizer. The use of a stabilizer in pharmaceutical compositions is well-known to the skilled person. For convenience reference is made to Remington: The Science and Practice of Pharmacy, 20^{th} edition, 2000.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or mixtures thereof) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or mixtures thereof) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives-(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [749]-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a OGP fusion protein according to the pre-sent invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the OGP fusion protein, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of OGP fusion protein, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the OGP fusion protein in the form of a nasal or pulmonal spray. As a still further option, the pharmaceutical compositions containing the OGP fusion protein of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the OGP fusion protein is stable for more than 6 weeks of usage and for more than 3 years of storage.

In another embodiment of the invention the pharmaceutical formulation comprising the OGP fusion protein is stable for more than 4 weeks of usage and for more than 3 years of storage.

In a further embodiment of the invention the pharmaceutical formulation comprising the OGP fusion protein is stable for more than 4 weeks of usage and for more than two years of storage.

In an even further embodiment of the invention the pharmaceutical formulation comprising the OGP fusion protein is stable for more than 2 weeks of usage and for more than two years of storage.

### EXAMPLES

### Cloning

The following constructs have been made: OGP-hGH (pNNC37) OGP(1-9)-hGH (pNNC37.1), OGP-OGP-hGH (pNNC38), OGP(1-9)-OGP(1-9)-hGH (pNNC38.1), OGP-OGP-OGP-hGH (pNNC38.2), OGP-OGP-NDEMPADLPS-hGH (pNNC39) and OGP(1-9)-OGP(1-9)—NDEMPADLPS-hGH (pNNC39.1).

Since OGP and variants thereof according to the present invention are fairly short peptides the cloning of OGP-hGH, OGP(1-9)-hGH, OGP-OGP-hGH and OGP(1-9)-OGP(1-9)-hGH utilized OGP encoding DNA oligo linkers for direct cloning into the Nde1 and Sal1 site in the pNNC19 bacterial expression vector that already contains the human growth hormone gene (pNNC19 is based on the commercial available pET11a vector). The OGP encoding sequences was codon optimized for E. coli expression using the Vector Suit NTI programme. When dimeric or trimeric OGP sequences are used, alternate codon usage may be utilized in the repeat to avoid genetic instability and cross hybridization. The oligos containing the dimeric forms of OGP have been cloned into pNNC19 as two segments. The cloning strategy to generate a OGP-OGP-OGP-hGH encoding vector (pNNC38.2) utilizes the previously generated OGP-OGP-hGH encoding vector pNNC38. The pNNC38 vector was cut with the cutting restriction enzymes Xba1 and Nde1, and a new DNA oligo linker containing an extra OGP(1-13) sequence added to vector. Since Nde1 site is right in front of the start codon (of OGP-OGP-hGH), this codon was subsequently changed into a glycine encoding codon by site directed mutagenesis creating an additional full length OGP(1-14) encoding sequence.

The following two constructs containing an albumine spacer sequence have been prepared: OGP-OGP-NDEMPADLPS-hGH and OGP(1-9)-OGP(1-9)-NDEMPADLPS-hGH. To obtain these constructs, a PCR based approach utilized the existing pNNC38 or pNNC38.1 as template and two independent PCR reactions each utilizing primers containing the NDEMPADLPS encoding sequence. The two NDEMPADLPS encoding PCR amplicons were cut with SacII to generate sticky ends and cloned back to the parental pNNC38 vector using the cutter sites Sph1 and BamH1 to generate pNNC39 encoding OGP-OGP-NDEMPADLPS-hGH and pNNC39.1 encoding OGP(1-9)-OGP(1-9)-NDEMPADLPS-hGH. The sequence of all constructs mentioned above have been confirmed by DNA sequencing of the fusion protein encoding region.

The oligo for the construction of OGP-hGH containing vector is shown in SEQ ID NO:5. This sequence comprises a sequence encoding OGP with an additional N-terminal methionine, a sequence encoding the first 6 amino acids in hGH and restriction sites in both ends.

The oligo for the construction of OGP(1-9)-hGH is shown in SEQ ID NO: 6. This sequence comprises a sequence encoding OGP(1-9) with an additional N-terminal methionine, a sequence encoding the first 6 amino acids in hGH and restriction sites in both ends.

The oligo for the construction of OGP-OGP-hGH is shown in SEQ ID NO: 7. To reduce the possible mutations generated in the DNA oligo synthesis the ligation was performed using a combination of four short oligos shown in SEQ ID NO: 8 to SEQ ID NO: 11

The oligo linker for the construction of OGP(1-9)-OGP(1-9)-hGH is shown in SEQ ID NO: 12. This sequence comprises two sequences encoding OGP (1-9) (one of them being the linker), a sequence encoding the first 6 amino acids of hGH and restriction sites in both ends. To reduce the possible mutations generated in the DNA oligo synthesis the ligation was performed using a combination of four short oligos shown in SEQ ID NO:13 to SEQ ID NO:16.

The DNA oligo linkers for the construct of OGP-OGP-OGP-hGH are shown is SEQ ID NO:21 and 22, and the primers for site directed mutagenesis are shown in SEQ ID NO:23 and 24.

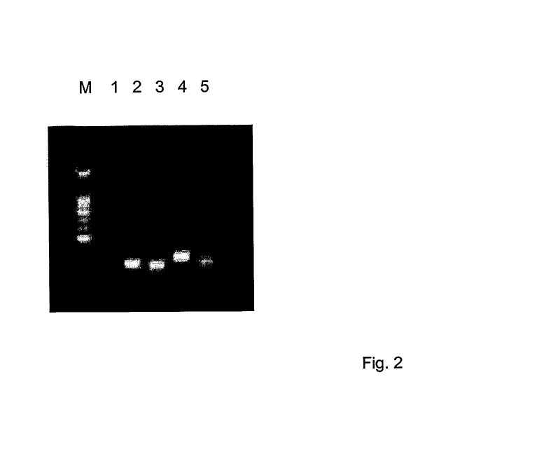

The OGP containing DNA oligo linkers were phosphorylated using T4 polynucleotide kinase (PNK) in standard buffer. NaCl and EDTA were added, the temperature was raised to 95° C. and the mixture was allowed to cool slowly to room temperature. The heating inactivates PNK and facilitate oligo annealing. Purified Nde1 and Sal1 cut pNNC19 vector was mixed with the OGP encoding oligos and allowed to ligate over night using T4 DNA ligase. Competent bacteria was transformed with the ligated vectors and positive clones detected using a diagnostic polymerase chain reaction (PCR) utilizing an OGP specific primer together with growth hormone specific primer (see FIG. 2).

The following PCR primers were used to generated OGP-OGP-NDEMPADLPS-hGH:

The following PCR primers were used to generated
OGP-OGP-NDEMPADLPS-hGH:

(SEQ ID NO:25)

SphI-F GAATGGTGCATGCAAGGAGATGGCGCCCAA;

(SEQ ID NO:26)

SacII-R 20GP GCAGATCCGCGGGCATTTCATCGTT GCCACCAAAG-
CCATACAGCGTGCGG;

(SEQ ID NO:27)

SacII-F AAATGCCCGCGGATCTGCCGAGC TTCCCGACCATCCCGCTG
AGTCG;
and

(SEQ ID NO:28)

BamHI-R AGCCGGATCCCTAGAAGCCACAGCTGCCCT.
The following PCR primers were used to generated
OGP(1-9)-OGP(1-9)-NDEMPADLPS-hGH:

(SEQ ID NO:29)

SphI-F GAATGGTGCATGCAAGGAGATGGCGCCCAA;

(SEQ ID NO:30)

SacII-R 20GP-D GCAGATCCGCGGGCATTTCATCGTTCAGCGTGCG-
GCCTTGGCGCTTCAGG;

(SEQ ID NO:31)

SacII-F AAATGCCCGCGGATCTGCCGAGC TTCCCGACCATCCCGCTG
AGTCG;
and

(SEQ ID NO:32)

BamHI-R AGCCGGATCCCTAGAAGCCACAGCTGCCCT.

Expression

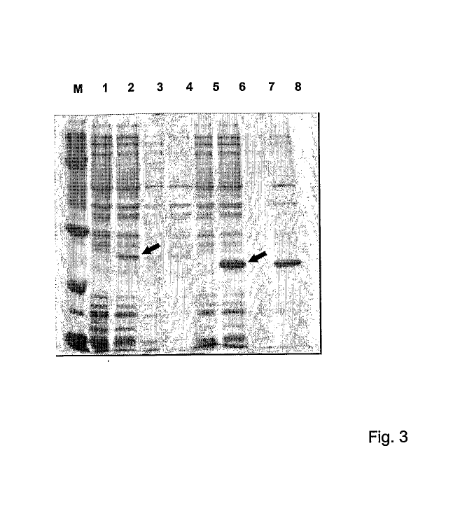

For protein expression BL21 bacteria were transformed with the above mentioned plasmids, grown to an OD₆₀₀ of approximately 0.6 and expression induced with 0.1 mM IPTG. After four hours the protein expression was analysed on SDS PAGE gels (see FIG. 3)

Purification

Anion exchange (DEAE-sepharose FF) can be used for OGP-hGH and OGP(1-9)-hGH with an expected pl around 6.0. Cation exchange (S-sepharose FF) can be used for OGP-OGP-hGH and OGP(1-9)-OGP(1-9)-hGH which are expected to have a higher pl around 7.8. The samples can follow the same route of chromatography purification as follows: Hydrophobic interaction (Phenyl Sepharose 6 FF), ion exchange, gel filtration (Sephadex G25), and freeze-drying.

OGP-hGH Degradation

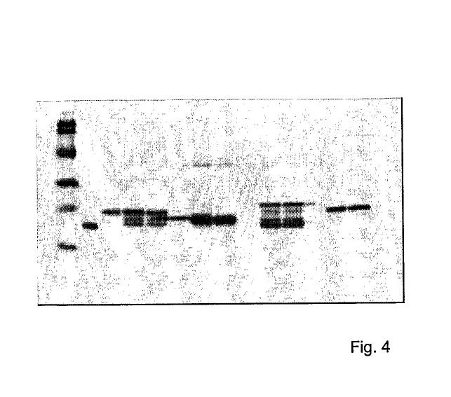

Western blot of the E. coli expression of the soluble form of the OGP-constructs shows that for OGP-hGH and OGP-OGP-hGH degradation products can be detected after cell lysis, see FIG. 4. However, for OGP(1-9)-hGH and OGP(1-9)-OGP(1-9)-hGH, no degradation is detected, indicating some degradation within the OGP C-terminal osteogeneic pentapeptide. The degradation products were analysed by in-gel trypsin digest followed by mass spectrometry, and it was shown that the degradation occurs at three specific sites in the pentapeptide. Temperature control during cell lysis and addition of protease inhibitors to the lysis buffer could not fully prevent degradation of the full length OGP constructs. The expression of these proteins in inclusion bodies is thus a more suitable process. In addition, we have shown that the intrinsic ability of E. coli to remove the N-terminal methionine occurs in OGP fusion proteins.

Refolding and Purification of OGP-hGH and OGP(1-9)-hGH

Inclusion bodies of OGP-hGH and OGP(1-9)-hGH are solubilised in 8 M Urea, 20 mM DTT, 20 mM Tris pH 9.0 at a concentration of 10 mg/ml and refolded by 50-fold dilution refolding in 20 mM Tris pH 9.0, 0.05% Tween-20 at 4° C. over night. First purification step is performed on a Q Sepharose FF column (buffer A; 20 mM Tris pH 9.0, buffer B; 20 mM Tris pH 9.0, 1 M NaCl). The protein is eluted by gradient elution.

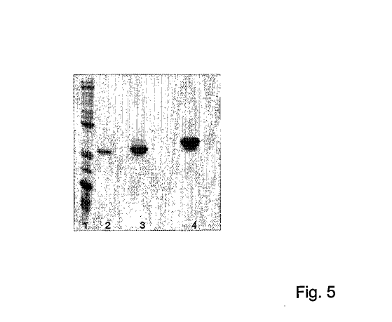

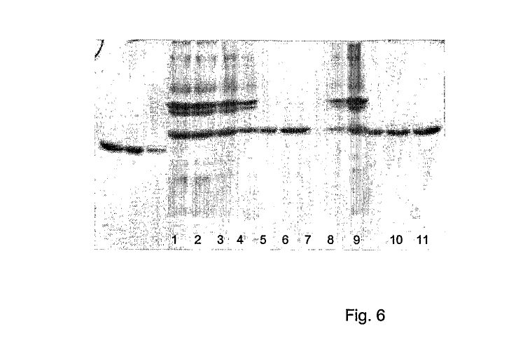

As the protein preparations contain dimers and other oligomeric forms of the proteins, gel filtration on a pre-packed HiLoad 26/60 Superdex 75 prep grade column (Amersham Biosciences) is performed as the second and final purification step. Buffer; 50 mM NH₄HCO₃ pH 7.8. The purity of the final OGP-hGH protein pool is illustrated in FIG. 5. Refolding and purification of OGP-OGP-hGH OGP-OGP-hGH was refolded by 50-fold dilution refolding in 1 M NDSB201, 1 M Urea and 20 mM Tris pH 6.0. Purification was performed on an SP Sepharose FF column (buffer A; 50 mM Na₂HPO₄ 50 mM NaH₂PO₄ pH 6.0, buffer B; 50 mM Na₂HPO₄ 50 mM NaH₂PO₄ pH 6.0, 1 M NaCl). The protein was eluted by step elution. The purity is illustrated in FIG. 6.

Purification of OGP(1-9)-OGP(1-9)-hGH

OGP(1-9)-OGP(1-9)-hGH is expressed in the soluble form. The pellet from E. coli expression is dissolved in lysis buffer (50 mM Na₂HPO₄ 50 mM NaH₂PO₄ pH 6.0, 5 mM EDTA, 0.1% Triton X-100) and cells are lysed by cell disruption at 30 kpsi. The supernatant is used for purification on an SP Sepharose FF column (buffer A; 50 mM Na₂HPO₄ 50 mM NaH₂PO₄ pH 6.0, buffer B; 50 mM Na₂HPO₄ 50 mM NaH₂PO₄ pH 6.0, 1 M NaCl). The protein is eluted by buffer B; 50 mM Na₂HPO₄ 50 mM NaH₂PO₄ pH 6.0, 1 M NaCl). The protein is eluted by step elution.

Refolding and Purification of OGP-OGP-OGP-hGH

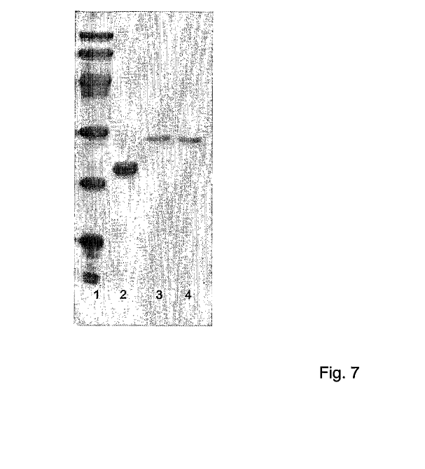

OGP-OGP-OGP-hGH can not be refolded by 50-fold dilution of the solubilised inclusion bodies and is therefore refolded on a HiLoad 16/60 Superdex 75 prep grade column (Amersham Biosciences) using a urea gradient (buffer A; 1 M Urea, 20 mM Tris pH 7.5, buffer B; 8 M Urea, 20 mM Tris pH 7.5). The column is equilibrated in 0-100% buffer B over 1 CV before loading of the sample. Further purification is performed on HiTrap CM Sepharose FF (buffer A; 25 mM K₂HPO₄-KH₂PO₄ pH 8, buffer B; 25 mM K₂HPO₄-KH₂PO₄ pH 7.1, 1 M NaCl). 3OGP-hGH is in the flow through. The SDS-PAGE in FIG. 7 shows a OGP-OGP-OGP-hGH preparation.

Surface Plasmon Resonance Analysis

Binding of hGH-OGP fusion proteins to α₂-macroglobulin was analyzed by surface plasmon resonance in a Biacore 3000 Instrument (Biacore AB, Uppsala, Sweden) essentially as described elsewhere [Biochemistry, 39(35), 10627-10633, 2000]. Briefly, α₂-macroglobulin (American Diagnostica Inc., Stamford, Conn.) at 20 μg/ml in 10 mM sodium acetate, pH 5.0 was immobilized (7 min at 5 μl/min) in flow cell 2 of a CM5 Biacore sensor chip which had been pre-activated with EDC/NHS using Amine Coupling Kit according to manufacturer's recommendations (Biacore AB, Uppsala, Sweden). Following protein immobilization, the surface was blocked by exposure to 1 M ethanolamine for 7 min at 5 μl/min. The final coupling yield was 23 fmol/mm². Kinetic analysis was performed at a flow rate of 10 μl/min in running buffer (10 mM HEPES, 150 mM NaCl, 5 mM CaCl₂, 0.05% Tween 20, pH 7.4) using the untreated flow cell 1 for automatic in-line reference subtraction. Following 5 min equilibration of the flow cells in running buffer, 100 μl protein sample was injected using the KINJECT command. The dissociation phase lasted 9 min and regeneration was performed with a 1-min pulse of 10 mM glycine, 500 mM NaCl, 20 mM EDTA, pH 6.0. SPR data were analyzed using BIAevaluation 4.1 software (Biacore AB, Uppsala, Sweden).

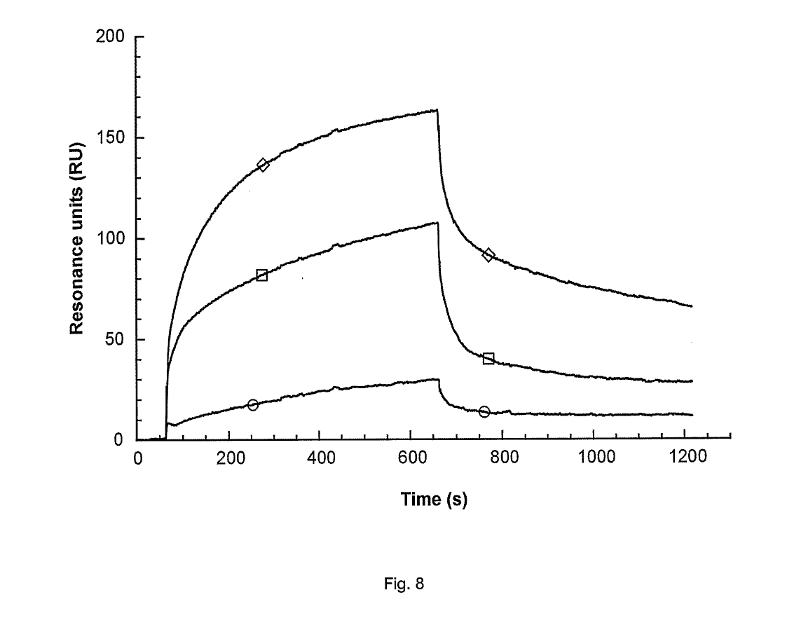

A fit of the sensorgrams to a 1:1 Langmuir binding model using BIAevaluation 4.1 software yielded apparent dissociation constants (K_{D}) of 210 nM and 1 μM for binding of OGP-OGP-hGH and OGP-hGH, respectively, to α₂-macroglobulin. See FIG. 8. Pharmacokinetics of 3OGP-hGH and 2OGP-NDEMPADLPS-hGH derivates after single dose iv and sc administration to rats.

Design

The study is performed in 18 Spraque-Dawley male rats weighing from 200 to 300 g. The animals are separated into four groups, see table 1.

TABLE 1
Treatment

| Compound | Animal No | Administration | Dose |
|---|---|---|---|
| 3OGP-hGH | 1-5 | SC | 1 mg/kg |
| | 6-9 | IV | 1 mg/kg |
| 2OGP- | 10-14 | SC | 1 mg/kg |
| NDEMPADLPS-hGH | 15-18 | IV | 1 mg/kg |

The test substance is dosed intravenously in a tail vein or subcutaneously in the neck with a 25 G needle.

Pharmacokinetic analysis is performed according to procedures known in the art. The analysis is carried out by non-compartmental methods using the software WinNonlin Professional, version 4.1 (Pharsight Corporation, USA).

Pharmacological Methods

Assay (I) Growth Activity in a BAF Assay

Murine lymphoid cells derived from bone marrow were transfected with cloned human growth hormone receptor. The cells are hence dependent on growth hormone for growth and survival. Cells were starved for growth hormone for 24 h, incubated with the test compounds for 3 days. Alamarblue colour-shift was the final result showing proliferation. The final results were calculated as relative to growth hormone. The relative potency of OGP-hGH and OGP-OGP-hGH was 78% and 67%, respectively.

1-28. (canceled)

29. A fusion protein comprising a first protein fused to the C-terminal of Osteogenic Growth Peptide (OGP) or a variant thereof, wherein, if said first protein is fused to OGP said first protein is not salmon calcitonin or OGP.

30. A fusion protein according to claim 29, wherein said fusion protein comprises a first protein fused to the C-terminal of OGP via a linker.

31. A fusion protein according to claim 30, wherein said linker is selected from the group consisting of: OGP, OGP-OGP, OGP(1-9), OGP(1-9)-OGP(1-9) and NDEMPADLPS.

32. A fusion protein according to claim 29, wherein said OGP variant differs from OGP by deletion of up to 5 amino acid residues of the C-terminus of OGP.

33. A fusion protein according to claim 32, wherein said OGP variant is OGP(1-9).

34. A fusion protein according to claim 29, wherein said first protein comprises human growth hormone or fragments thereof.

35. A fusion protein according to claim 29, selected from the group consisting of

| | |
|---|---|
| OGP-hGH,; | (SEQ ID NO:1) |
| OGP(1-9)-hGH,; | (SEQ ID NO:2) |
| OGP-OGP-hGH,; | (SEQ ID NO:3) |
| OGP(1-9)-OGP(1-9)-hGH; | (SEQ ID NO:4) |
| OGP(1-9)-OGP(1-9)-OGP(1-9)-hGH; | (SEQ ID NO:17) |
| OGP-OGP-OGP-hGH; | (SEQ ID NO:18) |
| OGP-OGP-NDEMPADLPS-hGH; | (SEQ ID NO:19) |
| and | |
| OGP(1-9)-OGP(1-9)-NDEMPADLPS-hGH | (SEQ ID NO:20) |

wherein hGH denotes human growth hormone.

36. A method of increasing circulation time of a protein, the method comprising producing a fusion protein according to claim 29.

37. An OGP variant, wherein up to five amino acids have been deleted from the C-terminal of OGP.

38. The OGP variant of claim 37 which is OGP(1-9).

39. An isolated nucleic acid construct comprising a nucleic acid sequence encoding a fusion protein as defined in claim 29.

40. A vector comprising the nucleic acid construct of claim 39.

41. A host cell comprising the nucleic acid construct of claim 39.

42. A method for producing a protein, said method comprising (i) culturing a host cell as defined in claim 41 under conditions suitable for expression of said nucleic acid construct and (ii) harvesting said protein from said culture.

43. A pharmaceutical composition comprising a fusion protein as defined in claim 29.

44. A method of treating a growth hormone-responsive syndrome, said method comprising the method comprising administering to a patient in need thereof a therapeutically effective amount of an OGP fusion protein according to claim 29.

45. A method as defined in claim 44, wherein said syndrome is selected from the group consisting of: growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones; fractures in or of spongious bones; tendon or ligament surgery; distraction oteogenesis; hip or discus replacement, meniscus repair, spinal fusions or prosthesis fixation; non-union or mal-union of fractures; osteatomia; graft implantation; articular cartilage degeneration in knee caused by trauma or arthritis; osteoporosis; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucucorticoid treatment in children,


SEQUENCE LISTING (34 sequences)

SEQ ID NO: 1 - protein, 205 aa, Artificial
  synthetic
ALKRQGRTLYGFGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - protein, 200 aa, Artificial
  synthetic
ALKRQGRTLFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 3 - protein, 219 aa, Artificial
  synthetic
ALKRQGRTLYGFGGALKRQGRTLYGFGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 4 - protein, 209 aa, Artificial
  synthetic
ALKRQGRTLALKRQGRTLFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 5 - DNA, 66 bp, Artificial
  synthetic
  exon (2)..(66)
tatggctctgaaacgtcagggtcgtaccctgtacggtttcggtggtttcccgaccatcccgctgag

SEQ ID NO: 6 - DNA, 51 bp, Artificial
  synthetic
  exon (2)..(51)
tatggctctgaaacgtcagggtcgtaccctgttcccgaccatcccgctgag

SEQ ID NO: 7 - DNA, 108 bp, Artificial
  synthetic
  exon (2)..(108)
tatggctctgaaacgtcagggtcgtaccctgtacggtttcggtggtgccctgaagcgccaaggccgcacgctgtatggctttggtggcttcccgaccatcccgctgag

SEQ ID NO: 8 - DNA, 64 bp, Artificial
  synthetic
tatggctctgaaacgtcagggtcgtaccctgtacggtttcggtggtgccctgaagcgccaaggc

SEQ ID NO: 9 - DNA, 44 bp, Artificial
  synthetic
cgcacgctgtatggctttggtggcttcccgaccatcccgctgag

SEQ ID NO: 10 - DNA, 66 bp, Artificial
  synthetic
tcgactcagcgggatggtcgggaagccaccaaagccatacagcgtgcggccttggcgcttcagggc

SEQ ID NO: 11 - DNA, 44 bp, Artificial
  synthetic
accaccgaaaccgtacagggtacgaccctgacgtttcagagcca

SEQ ID NO: 12 - DNA, 78 bp, Artificial
  synthetic
  exon (2)..(78)
tatggctctgaaacgtcagggtcgtaccctggccctgaagcgccaaggccgcacgctgttcccgaccatcccgctgag

SEQ ID NO: 13 - DNA, 44 bp, Artificial
  synthetic
tatggctctgaaacgtcagggtcgtaccctggccctgaagcgcc

SEQ ID NO: 14 - DNA, 34 bp, Artificial
  synthetic
aaggccgcacgctgttcccgaccatcccgctgag

SEQ ID NO: 15 - DNA, 29 bp, Artificial
  synthetic
cagggtacgaccctgacgtttcagagcca

SEQ ID NO: 16 - DNA, 51 bp, Artificial
  synthetic
tcgactcagcgggatggtcgggaacagcgtgcggccttggcgcttcagggc

SEQ ID NO: 17 - protein, 218 aa, Artificial
  synthetic
ALKRQGRTLALKRQGRTLALKRQGRTLFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 18 - protein, 233 aa, Artificial
  synthetic
ALKRQGRTLYGFGGALKRQGRTLYGFGGALKRQGRTLYGFGGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 19 - protein, 219 aa, Artificial
  synthetic
ALKRQGRTLALKRQGRTLNDEMPADLPSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 20 - protein, 229 aa, Artificial
  synthetic
ALKRQGRTLYGFGGALKRQGRTLYGFGGNDEMPADLPSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 21 - DNA, 82 bp, Artificial
  synthetic
ctagaaataattttgtttaactttaagaaggagatatacatatggcgttgaaacgtcaggggaggactttgtacggattcgg

SEQ ID NO: 22 - DNA, 80 bp, Artificial
  synthetic
taccgaatccgtacaaagtcctcccctgacgtttcaacgccatatgtatatctccttcttaaagttaaacaaaattattt

SEQ ID NO: 23 - DNA, 39 bp, Artificial
  primer
actttgtacggattcggtggagctctgaaacgtcagggt

SEQ ID NO: 24 - DNA, 39 bp, Artificial
  primer
accctgacgtttcagagctccaccgaatccgtacaaagt

SEQ ID NO: 25 - DNA, 30 bp, Artificial
  primer
gaatggtgcatgcaaggagatggcgcccaa

SEQ ID NO: 26 - DNA, 50 bp, Artificial
  primer
gcagatccgcgggcatttcatcgttgccaccaaagccatacagcgtgcgg

SEQ ID NO: 27 - DNA, 46 bp, Artificial
  primer
aaatgcccgcggatctgccgagcttcccgaccatcccgctgagtcg

SEQ ID NO: 28 - DNA, 30 bp, Artificial
  primer
agccggatccctagaagccacagctgccct

SEQ ID NO: 29 - DNA, 30 bp, Artificial; identical to SEQ ID NO: 25
  primer

SEQ ID NO: 30 - DNA, 50 bp, Artificial
  primer
gcagatccgcgggcatttcatcgttcagcgtgcggccttggcgcttcagg

SEQ ID NO: 31 - DNA, 46 bp, Artificial; identical to SEQ ID NO: 27
  primer

SEQ ID NO: 32 - DNA, 30 bp, Artificial; identical to SEQ ID NO: 28
  primer

SEQ ID NO: 33 - protein, 14 aa, Artificial
  synthetic
ALKRQGRTLYGFGG

SEQ ID NO: 34 - protein, 10 aa, Artificial
  synthetic
NDEMPADLPS